(19) United States
(12) Patent Application Publication
(10) Pub. No.: US 2001/0021531 A1
(43) Pub. Date: Sep. 13, 2001

(54) Food contamination detection system for vacuum packaged food products

(75) Inventor: Robert M. Goldsmith, Pasadena, CA (US)

Correspondence Address: LYON & LYON LLP, SUITE 4700, 633 WEST FIFTH STREET, LOS ANGELES, CA, 90071-2066, US

(73) Assignee: Sira Technologies, Pasadena, CA (US)

(21) Appl. No.: 09/855,029

(22) Filed: May 14, 2001

Related U.S. Application Data
(63) Continuation of application No. 09/489,080, filed on Jan. 21, 2000.

(51) Int. Cl. G01N 33/02

(52) U.S. Cl. 436/1; 436/20; 422/56; 426/87; 426/232

(57) ABSTRACT

The disclosure describes food contamination detectors adapted to be used with vacuum packages and package systems including such detectors.

### CROSS REFERENCE TO RELATED APPLICATIONS

This application is a continuation of U.S. application Ser. No. 09/489,080, filed on Jan. 21, 2000. The foregoing application is hereby incorporated by reference as if set forth fully herein.

### BACKGROUND OF THE INVENTION

The field of the present invention is the detection of substances or contaminants in edible consumer products.

Over the past several years there has been increasing concern over the safety of our food supply. Contamination of food can come from a variety of sources and the type of contamination possible is often dependent on the type of food involved.

Most animal derived food products, such as raw meat, are exposed to contamination before, during, or after processing. In most cases, contamination is minor and, if the food is prepared properly, may not pose a serious threat to the consumer. However, while the contamination of food is generally low, i.e. few bacteria per gram of food, if the food is not stored under satisfactory conditions, or if the food is stored for long periods of time, contaminants, such as bacteria, may grow to become a serious threat to the eventual consumer. Even if the food products reach the market in an acceptable condition, subsequent mistreatment by the consumer may lead to the development of food contamination.

A number of incidents and factors have lead to the growing concern over the food supply. These include:

raw chicken and egg products have been found to be contaminated with Salmonella and inadequate cooking of such products has led to serious illness or death of persons who have consumed the contaminated products;

inadequately pasteurized milk products have been found to be contaminated with Listeria which has lead to serious illness or death of consumers of the products;

a highly toxic stain of E. coli has lead to the death of several people who consumed prepared beef products which had been inadequately cooked;

a number of toxins are known, such as ciguatoxins, which contaminate fish. These toxins are not inactivated or destroyed by cooking and so their presence in fish is a threat to any consumer of the product;

shell fish, such as oysters, concentrate contaminants present in the water in which they grow and, since they are frequently eaten raw, pose a threat to the health of consumers; and

fish are increasingly eaten raw which adds to the possibility of increased outbreaks of illness from water borne contaminants.

Food products are often “mass produced” and sold at retail outlets in prepackaged containers. Such packages typically include a styrofoam, plastic or cardboard tray which supports the food product. The tray and food are sealed in a transparent plastic wrap material and a liner lies between the food product and the inside bottom of the tray. A bar code is often used on the products for scanning at the check-out register, to reduce errors in totaling purchases and for stock control. The bar code comprises a series or pattern of bars, which represent a number identifying the product.

The food industry is presently shifting to hermetically sealed, vacuum packaged food products which include fish, meats, poultry and other edible products. The present invention serves to enhance the detection of contaminants by directional capillary flow detection systems (described, for example, in U.S. application Ser. No. 09/153,562, filed Sep. 15, 1998) when these are used with hermetically sealed, vacuum packaged food products.

U.S. application Ser. No. 09/153,562 filed Sep. 15, 1998 and co-pending U.S. application Ser. Nos. 08/584,984 filed Jan. 11, 1996, Ser. No. 08/197,297 filed Feb. 16, 1994, and Ser. No. 08/758,205 filed Nov. 26, 1996 and U.S. Pat. No. 5,306,466 issued Apr. 26, 1994 (all of which are incorporated by reference as if fully set forth herein) describe inventions for the detection of contaminants in food.

### SUMMARY OF THE INVENTION

A first, separate aspect of the present invention, is directed at a venting arrangement that enhances the directional capillary flow of fluids in detection systems when used with hermetically sealed, vacuum packed food products.

A second, separate aspect of the present invention, is directed at a food package including a venting arrangement associated with a capillary contamination detector, and to methods of detecting contamination in food contained within packages that include the invention.

A third, separate aspect of the present invention, is directed at a switch that initiates food contamination detection.

In a fourth, separate aspect of the present invention, the switch includes a hermetic seal that when removed activates the venting arrangement, which in turn initiates the capillary flow of juices through the detection system.

### BRIEF DESCRIPTION OF THE DRAWINGS

FIG. 1 is a longitudinal cross sectional view of a contamination detector for use in food safety according to a preferred embodiment.

FIGS. 2 and 3 illustrate a second embodiment of the present invention.

FIGS. 4 and 5 are schematics (top and bottom view, respectively) illustrating food packages of a kind which may incorporate detection systems including the present invention.

FIG. 6 illustrates a food package having a contamination detection system that incorporates the present invention.

FIG. 7 illustrates a package including a second embodiment of the present invention.

### DETAILED DESCRIPTION OF THE PREFERRED EMBODIMENT

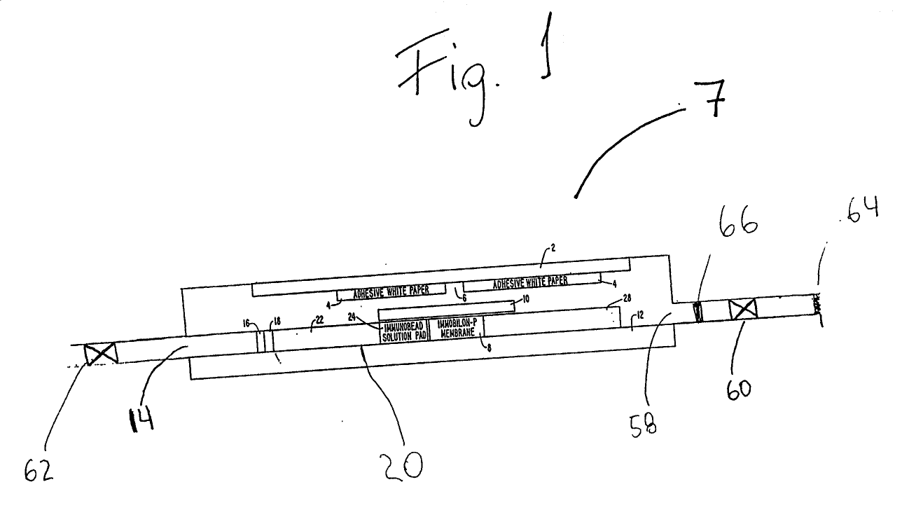

FIG. 1 is a longitudinal cross sectional view of a preferred embodiment of a contamination detector incorporating the features of the present invention. The figure shows a directional capillary flow contamination detection system 7 (U.S. application Ser. No. 09/153,562, filed Sep. 15, 1998, describes such systems and they construction in detail; any effective system, however, may be used in accordance with the present invention), which in addition incorporates components of the present invention.

As shown in FIG. 1, a cover film 2 composed of mylar acetate or some other type of transparent nonporous film contains a code indicia (not shown) such as the ubiquitous UPC bar code which codes for a particular food product that is contained in the package in which the particular contamination detection system is associated. The bars of the bar code may be printed onto the film and the light reflective spaces may be created by using adhesive white paper 4 or some other method such as painting or staining the back of the cover film 2, provided that light may be reflected to a bar code reading device such as a laser bar code scanner, light pen, CCD or other such device that can detect the bars and spaces of the bar code.

In the case of a bar code, the cover film 2 preferably has a window 6 which contains at least one space of the bar code. Window 6 is aligned with detection area (not shown) of substrate 8. Typically, the prepackaged food product will be shipped to a retail outlet such as a grocery store which will attach its own bar code label (cover film 2) to and in alignment with a detection area (not shown). When the contamination detection system detects contamination, the light reflectivity of detection area will change from being light reflecting to light absorbing, or vice-versa. Typically, for many UPC bar codes that means a color change from white to black. However, as known by those of ordinary skill in the art of bar code scanning, other colors, shades, or metallic lusters may be used to accomplish this effect. Once detection area becomes light absorbing, a bar code reading device will be unable to successfully decode the code indicia of cover film 2 and contamination can be detected.

Further detection methods that may be used with the present invention are described in the above referenced patent and patent applications. For example, these may include symbols, letters, words, and the like, which become readable or unreadable (depending on the implementation), in the presence of the antigen or antigens to be detected. In particular, the indicator area may be placed such that it obliterates part of the product identification code rendering it incomplete, or unreadable, to avoid the accidental checkout of contaminated product, or it may modify it, such that it represents a different code. One or several indicator areas may be present in a substrate 8. Alternatively, the code indicia may be characterized by utilizing, with the indicator, a first bar code for which the change in appearance of the indicator in the presence of antigen makes the first bar code unreadable by a bar code reader, and a second bar code for which the change in appearance of the indicator in the presence of antigen makes the second bar code readable by a bar code reader. Additionally, the indicator area could include a number which appears upon contamination to complete a numeric code corresponding to the data code by the contamination detecting bar code. With these additional elements, a checkout clerk could not inadvertently price contaminated product when bar code is not recognized or read by the scanner.

Above substrate 8 is migration film 10 which is composed of mylar acetate or some other transparent nonporous film. Below substrate 8 is a support 12 which can be a plastic strip, a film such as mylar acetate or some other nonporous film or substance. These two components ensure that liquids pass through the contamination detection system.

Prior to and abutting the absorbing material 22, a protective filter 16 may be present to filter out larger materials, which depending on the implementation may range from proteins to clumped dead cells, or the like. A selective filter 18, may also be present to filters out other materials and molecules larger than the toxin of interest such as ciguatoxin, brevetoxin or other toxins associated with food poisoning, or a bacteria of interest such as E. coli, Salmonella, Listeria, Campylobacter or other bacteria associated with food poisoning. Moreover, other means of filtering, well known in the art, may be used.

Absorbent pad 20 includes a primary absorbent material 22. As used herein, “absorbent pad” or “absorbent material” means any material providing for the directional capillary flow of juices or fluids. These materials include, for example, blotting paper, Whatman paper, structures having thin capillary conduits made of any of the several well known in the art materials that do not detrimentally affect the sample travelling therein, and the like. After the filtered liquid passes through the primary absorbent material 22, it passes through the immunobead solution pad 24. Immunobead solution pad 24 contains monoclonal antibodies bound to colored latex microspheres, and is created by standard techniques, as described in U.S. application Ser. No. 09/153,562 for example. As the filtered liquid passes through the immunobead solution pad 24, antigens bind to monoclonal antibodies that recognize the specific antigen. Advantageously, the immunobead solution pad may have one type of monoclonal antibody or multiple types of monoclonal antibodies to ensure detection of a single antigen. Alternatively, the immunobead solution pad may have multiple types of monoclonal antibodies to detect multiple antigens. Alternatively, the immunobead solution pad may use polyclonal antibodies instead of and/or in addition to monoclonal antibodies.

The preparation of such antigen-specific antibodies is well known in the art. In some cases it may be necessary to conjugate a toxin antigen to a protein to “mask” the toxicity of the antigen. Otherwise injection of the toxic antigen may result in the death of the animal in which the antibodies are to be prepared. Methods of conjugating compounds are well known in the art and one such method is described by Hokama et al., Mycotoxins and Phycotoxins 188, A Collection of Invited Papers at the Seventh International IUPAC Symposium of Mycotoxins and Phycotoxins, Tokyo, Japan 1988, pp. 303-310 (Elsevier Science Publishers, Amsterdam), which is incorporated herein by reference.

A substrate 8 adjoins the immunobead solution pad 24. Substrate 8 is a membrane, such as IMMOBILON-P, having a monoclonal antibody, or the like, bound to a detection area. Alternatively, a polyclonal antibody may be used on substrate 8 to form the detection area. The detection area may encompass the entire area of the substrate 8, or only a portion of it. Antibodies on the substrate 8 can be specific to the antibodies contained in the immunobead solution pad, the antigen or antigens, the antigen to be detected bound to the antibodies contained in the immunobead solution pad, or mixtures thereof. Although the first and second antibodies could be the same, they are preferably different. The second antibody preferably recognizes different antigenic determinants on the antigen than the first antibody. Consequently, when in use, as the filtered liquid passes across substrate 8, the antigens bound to antibodies bound to the colored latex microsphere are captured in the detection area. As the number of antibodies bound to the colored latex microsphere and a toxin or bacteria increases, so does the color of the detection area so that eventually the light reflectivity of detection area changes from light reflecting to light absorbing at which point contamination is considered to have occurred. The filtered liquid is then withdrawn by a secondary absorbent material 28.

In the embodiment shown in FIG. 1, an outlet 58, permits gases and other fluid materials to exit the detection system. A one way valve 60, is present such that gases and other fluids may exit the detection system, but not enter it. Although any one way valve may be used, preferred are valves that are effective at the low temperatures at which perishable food products are generally kept. Mechanical valves, such as described in U.S. Pat. No. 4,890,637, may be used for this purpose. Other suitable valves may also be used.

A removable cover seal 64, preferably hermetically seals the detector outlet 58. The seal serves to protect the system during transit of the packaged food, preventing particles and other elements from entering the outlet.

The removable cover seal 64, is also effective as a switch for initiating the detection of contaminants. In the embodiment where the removable cover seal 64 serves as a switch, the system inlet 14, has a length such that when the food juices travel through the inlet, a pressure is created within the detection system that blocks the front of travelling food juices. The system is preferably designed such that the front of travelling food juices is blocked before it reaches the immunobead solution pad 24. Removing the seal 64 permits gases to exit the contamination system through the outlet. The food juices, previously blocked by the pressure, are then free to progress through the detection system thus actuating the detection system.

The seal 64 may be of any effective material and may be constructed in any effective manner. The manufacture and use of such removable seals for use with vacuum packed food products is well know in the art under different terms, including tear out sealing diaphragm, pull tab, membrane-type seal, and the like. The seals are normally heat sealed or adhesively overlaid across the orifice. The seal preferably has a tamper indicator.

In the preferred embodiment a membrane 66 spans the outlet. The membrane 66 has the ability under normal operating conditions to permit the passage of gas, but to prevent the passage of liquid. Such membranes are well known in the art. For example, thin expanded plastic membranes exhibit the properties of being both waterproof and air permeable. The membrane may be of any effective thickness, but generally membranes less than 2 mm. thick are preferred. The membrane preferably includes microscopically minute pores which are small enough to permit the passage of gases but not liquids such as water. Two well known examples of such plastics are expanded polyurethane films and polytetrafluorethylene. However any material having these general properties may be used.

The membrane 66 preferably is of such construction and is placed in the system such that it effectively prevents liquids from exiting the detection system. The membrane 66 may also serve to prevent juices from affecting the performance of the one way outlet valve 60, if one is present.

An inlet valve 62, or the like, may be present at or before the inlet. The inlet valve 62, permits food juices to enter the detection system, but prevents the back-flow of juices from the system into the package. A switching mechanism may also be associated with the inlet valve.

While the embodiment shown in FIG. 1 has the inlet valve 62, outlet 58, outlet valve 60, membrane 66 and removable seal 64, these components need not all be present. The components may be present individually or in different combinations. More then one inlet, inlet valve, outlet, outlet valve, membrane and/or removable seal may also be present. The position of the components also need not be as shown. The membrane 66, for example, may be placed before the outlet valve 60, as shown in the figure, after it, one in each position, and the like. The seal may also be placed in other positions, but is preferably placed as shown in the figure. Other effective variations of the arrangements, which are within the skill of the art, are also within the scope of the present invention.

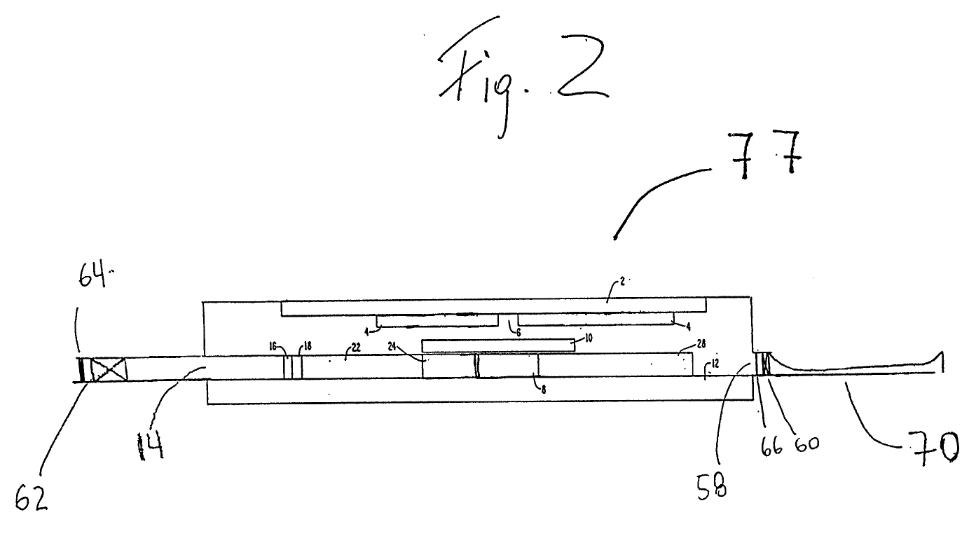

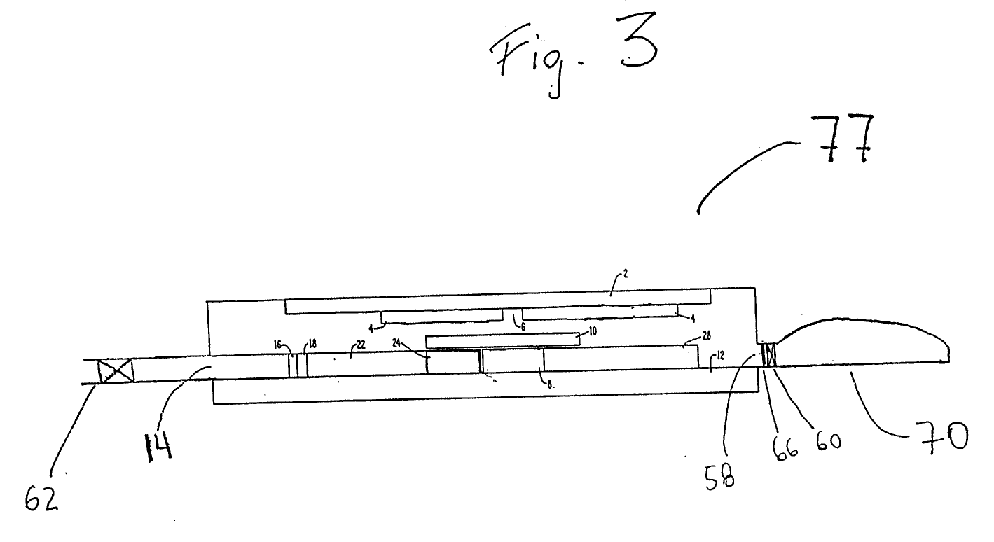

FIG. 2 illustrates another embodiment 77 of the present invention, wherein the outlet leads into a receiving zone 70, or gas collection bladder, which is preferably self contained. The receiving zone 70 may be of any suitable design, but preferably consists of an expandable and/or flexible bag like structure, which is initially empty. As the juice front travels through the detection system and displaces forward the few remaining gas molecules in the vacuum, the gas is displaced into the receiving zone 70, which then may expand as shown in FIG. 3. An advantage of the embodiment including the receiving zone 70 is that it obviates the need for an outlet that vents outside the vacuum package, therefore permitting the construction of a self contained system. An inlet valve or valves 62, outlet valve or valves 60, membrane or membranes 66 and seal or seals 64 may also optionally be present.

The receiving zone may also be available for the purpose of sampling entrapped gases by mechanical, optical, electronic, chemical or other analysis methods. The gases may be analyzed for any compounds of interest, for example to verify the presence of microorganisms. The receiving zone may be adhered to the interior of the package to facilitate sampling. The package exterior may be marked with a window or other demarcation alerting a technician to the appropriate location for gas sampling.

The contamination detection system described may be used in a preformed well of a food package as described in the above referenced patent and applications or, advantageously, it may be embedded in the food package itself. The food package is preferably of a type which permits vacuum packing. Alternatively, the contamination detection system may be embedded or closely associated with a liner, which commonly is present in the package of food products and lies between the tray and the food, such that juices collected in the liner are drawn into the contamination detection system.

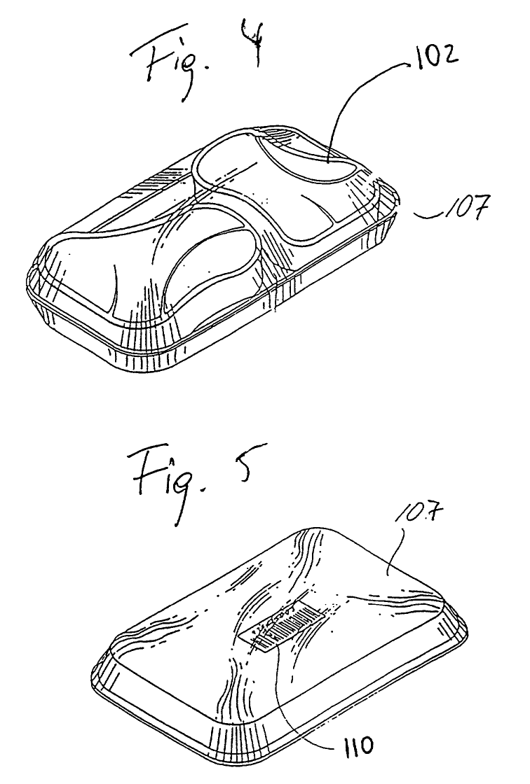

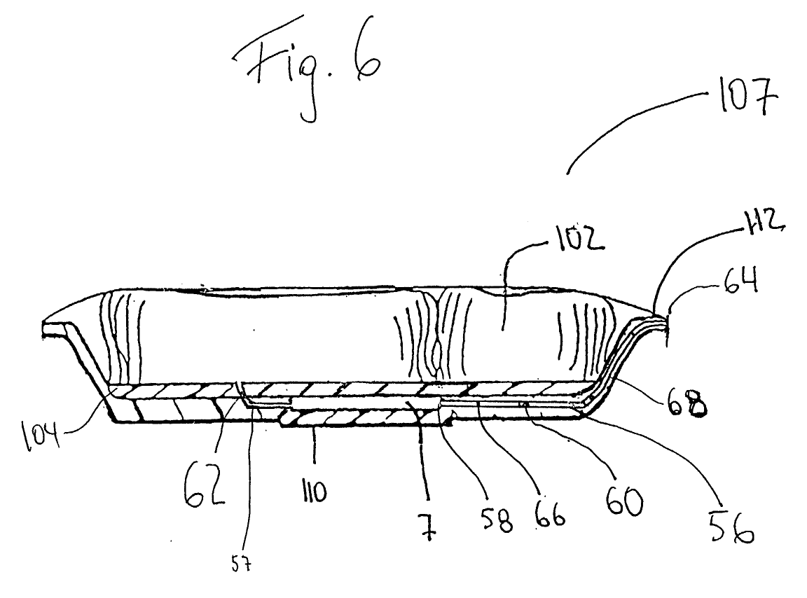

FIGS. 4 and 5 are schematics (top and bottom view, respectively) illustrating food packages of a kind which may incorporate detection systems including the present invention. See, e.g., WO 98/14777. An indicator 110 is illustrated in the package in FIG. 5. Food products 102 are stored within the package 107, preferably in a vacuum-packed format.

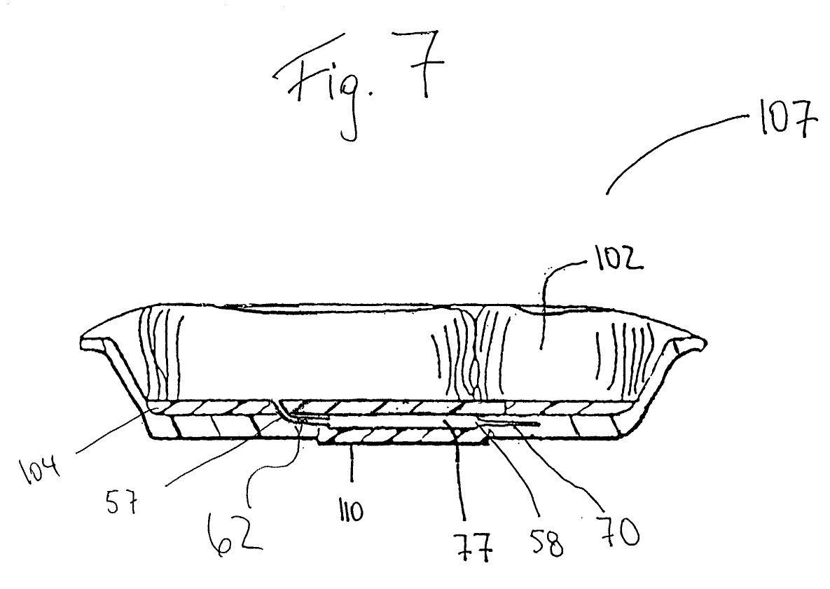

FIG. 6 illustrates a food package 107 having a contamination detection system 7, incorporating elements of the present invention, in particular the preferred embodiment illustrated in FIG. 1. Because of the capillary-action dependence of the detector 7, the detector 7 may be placed anywhere in the package. However, the detector 7 is preferably on the bottom of the package, abutting the indicator 110. An inlet valve 62, outlet 58, outlet valve 60, membrane 66 and removable seal 64 are illustrated in the outlet duct 56, but need not all be present, and if present need not be in that sequential order.

The inlet duct 57 may originate from any interior surface of the package, but is preferably in a position such that it may collect fluids from the package when the package is resting in a display shelf. The inlet duct 57, may, for example, collect fluids from a liner, if one is present in the package 107.

The outlet duct 56 may lead to any exterior surface of the package, but is preferably in a position such that it is higher than the indicator when the package is resting in a display shelf, such that liquids will not sip out of the outlet duct 56, once the seal, if one was present, is removed. Most preferably, the outlet duct 56, as illustrated in FIG. 6, leads to a lateral surface of an elevated lip 112 generally present in vacuum packages. Alternatively, it leads to the under-surface of the lip 112. Such positioning prevents blocking the outlet duct 56 when stacking the packages. Other positions achieving this result are also preferred. The duct is preferably sealed with a removable seal 64, as previously discussed.

A motion or position sensitive valve 68, or the like, may be present, such that juices or other liquids will not drip out of the outlet duct 56, when the package is handled. For example, a ball check valve 68, as illustrated, may be used such that the duct 56 is blocked when a customer turns the package 107 over to verify the condition of the indicator 110, 7.

FIG. 7 illustrates package 107 including a self contained indicator 77 as shown in FIGS. 2 and 3.

Thus, a food contamination detector adapted for use with vacuum packed food product has been disclosed. While embodiments and applications of this invention have been shown and described, it would be apparent to those skilled in the art that many more modifications are possible without departing from the inventive concepts herein. The invention, therefore is not to be restricted except in the spirit of the appended claims.

1. A food contamination detector for use with vacuum-packed food products comprising
a directional capillary flow contamination detection system having an inlet and an outlet;
a one way outlet vent in fluid communication with the outlet, wherein the outlet vent permits gases to exit, but not enter, the contamination detection system.

2. The food contamination detector of claim 1, further comprising
a removable cover seal hermetically attached to the detector outlet.

3. The food contamination detector of claim 1, further comprising
a membrane spanning the outlet, wherein the membrane permits gases, but not liquids, to exit the contamination detection system.

4. The food contamination detector of claim 1, further comprising
an inlet valve in fluid communication with the inlet, wherein the inlet valve prevents the back-flow of product juices from the contamination detection system into the package.

5. A food contamination detector to be used with vacuum-packed food products, the food contamination detector comprising
a directional capillary flow contamination detection system having an inlet and an outlet;
a removable cover seal hermetically attached to the outlet.

6. A food contamination detector to be used with vacuum-packed food products, the food contamination detector comprising
a directional capillary flow contamination detection system having an inlet and an outlet;
a receiving zone in fluid communication with the outlet.

7. A package adapted for containing a food product that produces juice, comprising the food contamination detector of claim 1.

8. The food product package of claim 7, wherein the food contamination detector further comprises
a removable cover, hermetically sealing the detection system's outlet or outflow duct.

9. The food product package of claim 7, wherein the food contamination detector further comprises
a one way venting valve in fluid communication with the outlet, wherein the valve permits gases to exit, but not enter, the detector.

10. The food product package of claim 7, wherein the food contamination detector further comprises
a membrane spanning the outlet, wherein the membrane permits gases, but not liquids, to exit the contamination detection system.

11. A package adapted for containing a food product that produces juice, comprising the food contamination detector of claim 6.

12. A food contamination detector for use vacuum-packed food products comprising
a directional capillary flow contamination detection system having an inlet and an outlet;
a one way inlet valve in fluid communication with the inlet, wherein the inlet valve serves to prevent the back-flow of product juices from the contamination detection system into a food package;
a one way outlet valve in fluid communication with the outlet, wherein the outlet valve permits gases to exit, but not enter, the contamination detection system;
a membrane spanning the outlet, wherein the membrane permits gases, but not liquids, to exit the contamination detection system;
a removable cover seal hermetically attached to the detector outlet.

13. A package adapted for containing a food product that produces juice, comprising the food contamination detector of claim 12.
